(19) United States
(12) Patent Application Publication
(10) Pub. No.: US 2011/0177038 A1
(43) Pub. Date: Jul. 21, 2011

(54) Inhibition of Tumor Growth via Peroxiredoxin 3

(75) Inventors: Chi V. DANG, Baltimore, MD (US); Diane WONSEY, Concord, MA (US)

(73) Assignee: THE JOHNS HOPKINS UNIVERSITY, Baltimore, MD (US)

(21) Appl. No.: 12/028,441

(22) Filed: Feb. 8, 2008

Related U.S. Application Data
(63) Continuation of application No. 10/408,766, filed on Apr. 8, 2003, now abandoned.
(60) Provisional application No. 60/370,873, filed on Apr. 8, 2002.

(51) Int. Cl. A61K 35/12; C12N 5/09; A61K 31/7052; A61K 35/00

(52) U.S. Cl. 424/93.7; 435/375; 514/44 A

(57) ABSTRACT

Deregulated expression of the c-Myc transcription factor is found in a wide variety of human tumors. Because of this significant role in oncogenesis, considerable effort has been devoted to elucidating the molecular program initiated by deregulated c-myc expression. The primary transforming activity of Myc is thought to arise through transcriptional regulation of numerous target genes. Thus far, Myc target genes involved in mitochondrial function have not been characterized in depth. Here, we describe a nuclear c-Myc target gene, PRDX3, which encodes a mitochondrial protein of the peroxiredoxin gene family. Expression of PRDX3 is induced by the mycER system and is reduced in c-myc−/− cells. Chromatin immunoprecipitation analysis spanning the entire PRDX3 genomic sequence reveals that Myc binds preferentially to a 930-bp region surrounding exon 1. We show that PRDX3 is required for Myc-mediated proliferation, transformation, and apoptosis after glucose withdrawal. Results using mitochondria-specific fluorescent probes demonstrate that PRDX3 is essential for maintaining mitochondrial mass and membrane potential in transformed rat and human cells. These data provide evidence that PRDX3 is a c-Myc target gene that is required to maintain normal mitochondrial function.

This application claims priority to provisional U.S. Application Ser. No. 60/370,873, filed Apr. 8, 2002.

This invention was made using funds from the U.S. National Cancer Institute. Therefore, under the terms of R37CA51497, the U.S. government retains certain rights in the invention.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The invention relates to therapy and drug development for tumors.

### BACKGROUND OF THE INVENTION

The c-Myc transcription factor has been implicated in the control of a variety of cellular processes, including cell growth, cell-cycle progression, and apoptosis (1). The c-Myc protein is a member of the basic helix-loop-helix leucine zipper family of transcription factors. In cooperation with its heterodimerization partner Max, Myc binds DNA in a sequence-specific manner and activates transcription at E box elements with the consensus sequence 5′-CACGTG-3′. In an effort to dissect the molecular pathways regulated by Myc, several recent studies have focused on the use of microarray technology to identify the transcriptional targets of c-Myc (2-4). A coherent picture is beginning to emerge whereby Myc functions to accelerate multiple metabolic pathways, including amino acid and nucleotide synthesis, lipid metabolism, and glycolysis. Whether Myc also affects mitochondrial metabolism remains unclear.

Because mitochondria play a central role in energy production as well as the execution of cell death, they represent a potential site for the regulation of both proliferation and apoptosis. Therefore, Myc target genes encoding mitochondrial proteins could play a significant role in tumorigenesis.

PRDX3 was first identified as a putative c-Myc target gene by using representational difference analysis (RDA) to identify genes that were differentially expressed between Rat1a (R1a) fibroblasts and R1a fibroblasts stably overexpressing c-Myc (R1a-myc) under conditions of anchorage-independent growth (5). Originally cloned as a gene expressed during the differentiation of murine erythroleukemia cells (6), PRDX3 was subsequently shown to possess peroxide reductase activity (7). PRDX3 belongs to an expanding family of highly conserved proteins termed peroxiredoxins, which catalyze the reduction of peroxides in the presence of thioredoxin (8, 9). Members of this gene family have been shown to be involved in diverse cellular roles, including proliferation (10), apoptosis (11), and the response to oxidative stress (12). The bovine PRDX3 homolog, SP-22, localizes to mitochondria, and SP-22 expression is induced after exposure to peroxides or mitochondrial respiratory chain inhibitors (12). The potential role of PRDX3 in tumorigenesis has recently been examined in breast cancer, where elevated levels of PRDX3 protein were found in 79% of the cases examined (13).

### BRIEF SUMMARY OF THE INVENTION

According to a first embodiment of the invention a method is provided. An antisense construct comprising at least 15 nucleotides of a murine or human PRDX3 cDNA is delivered to a tumor cell. The tumor cell thereby expresses an antisense RNA molecule which is complementary to native PRDX3 mRNA.

According to a second embodiment of the invention an RNA interference construct comprising at least 19 nucleotides of a murine or human PRDX3 cDNA is delivered to a tumor cell. The tumor cell thereby expresses a double stranded RNA molecule one of whose strands is complementary to native PRDX3 mRNA.

A third embodiment of the invention is another method for inhibiting expression of PRDX3. An siRNA comprising a 19 to 21 bp duplex of a murine or human PRDX3 mRNA with 2 nt 3′ overhangs, is delivered to a tumor cells. PRDX3 mRNA produced by the tumor cell is thereby cleaved.

A fourth embodiment of the invention is a method which can be used in drug discovery. A test substance is contacted with c-MYC protein and a murine or human PRDX3 genomic DNA molecule comprising at least one of the E-boxes selected from the group consisting of: CACGTG, CATGCG, and CGCGTG. Binding of c-MYC protein to said DNA molecule is determined. A test substance which inhibits binding of c-MYC protein to said DNA molecule is identified.

A fifth embodiment of the invention is another method. An inhibitor of peroxiredoxin 3 enzyme activity is delivered to a tumor cell.

### BRIEF DESCRIPTION OF THE DRAWINGS

FIG. 1A-1G. PRDX3 is regulated by c-myc expression. (FIG. 1A) RNA from Rat 1a (R1a) fibroblasts or Rat1 a fibroblasts expressing ectopic c-Myc (R1a-myc). rpL32 is shown as a loading control. RNA was isolated from adherent cells (A) or nonadherent cells grown over a layer of agar (N). (FIG. 1B) PRDX3 expression in logarithmically growing c-myc^{+/+}, ^{+/-}, or ^{-/-} Rat1 fibroblasts. PRDX3 expression was calculated relative to vimentin. (FIG. 1C) Hepatic RNA from mice injected with either adenoviral LacZ or c-myc. Numbers represent days after injection with adenovirus. 18S RNA is shown as a loading control. (FIG. 1D) Schematic representation of the PRDX3 genomic locus. Exons are indicated by black boxes. Fragments analyzed for Myc binding are indicated by lettered black bars. The sole canonical E box is indicated in bold, and noncanonical E boxes (38, 39) in fragments C and D are also shown. (FIG. 1E) Ethidium bromide-stained gels of PCR products. (FIG. 1F) Sybr green analysis of PCR fragments evaluated for Myc binding. The absolute amount of DNA in each sample was calculated, and the average was plotted ±SD. (FIG. 1G) Relative mRNA levels for c-myc and PRDX3 during serum stimulation. Signals were normalized to the level of 18S RNA and plotted relative to the 0 h time point for each series.

FIG. 2A-2E. Effect of PRDX3 expression on doubling time, transformation, and apoptosis in R1a-myc cells. (FIG. 2A) Immunoblot analysis of cell lysates from R1 a-myc cells transfected with pSG5 empty vector, pSG5-PRDX3, or pSG5-PRDX3AS. (FIG. 2B) Growth curves of R1a-myc transfectants: pSG5 (□), PRDX3 (Δ), and PRDX3AS (◯). Doubling times were 10.4, 10.9, and 19.0 h, respectively. (FIG. 2C) Photomicrographs of methylcellulose colonies. (Bar=500 μM.) The bar graph represents the average colony number per 35-mm dish ±SD. (FIG. 2D) Tumor formation in nude mice. The average estimated tumor mass was plotted at 2, 3, and 4 weeks after injection ±SD (n=8). (FIG. 2E) Percentage of apoptotic cells 24 h after serum deprivation (light bars) or glucose deprivation (dark bars). The average ±SD of three experiments is shown.

FIG. 3A-3C. Effect of PRDX3 expression on doubling time and apoptosis in MCF7/ADR cells. Effect of PRDX3 expression on doubling time and apoptosis in MCF7/ADR cells. (FIG. 3A) Immunoblot analysis of cells lysates from MCF7/ADR cells transfected with pSG5, pSG5-PRDX3, or pSG5-PRDX3AS. (FIG. 3B) Growth curves of MCF7/ADR transfectants: pSG5 (□), PRDX3 (Δ), and PRDX3AS (◯). Doubling times were 43.0, 37.6, and 60.2 h, respectively. (FIG. 3C) Percentage of apoptotic cells 24 h after glucose withdrawal. The average ±SD of three separate experiments is shown.

FIG. 4A-4C. PRDX3 affects mitochondrial membrane integrity and morphology. (FIG. 4A) Histograms generated by FACS analysis of cells incubated with dye specific for cellular reactive oxygen species (DCF), mitochondrial mass (NAO), or mitochondrial membrane potential (DiOC₆): pSG5 (solid black line), PRDX3 (solid gray line), PRDX3AS (dotted line). (FIG. 4B) Transmission electron microscopy of R1a-myc-pSG5 and R1a-myc-PRDX3AS cells. (Bar=1 μM.) (FIG. 4C) Analysis of ROS after glucose deprivation. Cells were exposed to glucose-free media for 1.5 h before incubation with DCFH-DA.

FIGS. 5A through 5D. Northern analysis of PRDX3 in the mycER system and c-myc null cells. (FIG. 5A) Regulation of PRDX3 in the MycER system. Analysis of MycER cells was performed on 15 μg of RNA isolated from confluent cells treated with 10 μM cycloheximide (CHX), 0.25 μM tamoxifen (TM), or both CHX+TM for the indicated times. The blot was hybridized to a probe for 36B4 [Laborda, J. (1991) Nucleic Acids Res. 19, 3998] as a loading control. Fold change was calculated relative to the 0 hr time point for each series after normalization to 36B4. (FIG. 5B) Serum stimulation of HO15 (c-myc^{-/-}) and TGR (c-myc^{+/+}) cells. Confluent cells were cultured in 0.1% serum for 48 hr prior to stimulation with medium containing 10% serum. Total RNA was collected at the indicated time points, and 10 μg of each sample was analyzed. (FIG. 5C) Quantitation of PRDX3 expression in c-myc (+/+) and (−/−) cells. Fold change was calculated relative to the 0 hr time point in each series after normalization to 18S RNA, which was quantitated by analyzing the ethidium bromide-stained gel with labworks image analysis software (UVP). (FIG. 5D) Luciferase activity of PRDX3 sequences positive for Myc binding by ChIP analysis. A 930-bp region from human PRDX3 genomic DNA (spanning fragments B, C, and D in FIG. 1) was amplified by using the primers C3XmaI (5′-tgcccggggacacagtaatccacacaagg-3′; SEQ ID NO: 1) and E2XhoI (5′-tgctcgaggccaccgcactctgccggtt-3; SEQ ID NO: 2). The fragment was cloned into the pGL2-Promoter vector (Promega) and transfected into 60% confluent NIH 3T3 fibroblasts with Lipofectamine (Invitrogen). Transfections consisted of 400 ng of reporter construct and 5 ng of murine leukemia virus-long terminal repeat-driven plasmid expressing either wild-type Myc (MLV-myc) or a mutant Myc that lacks the helix-loop-helix region and transformation activity (MLVΔHLH). Transfections were performed in triplicate, and total DNA was normalized using pBluescript II SK(+).

### DETAILED DESCRIPTION OF THE INVENTION

It is a discovery of the present invention that PRDX3 is a direct target of cMyc. cMyc directly binds to specific portions of the PRDX3 gene and activates its transcription. Moreover, PRDX3 mediates at least some of the functions of cMyc which are involved in tumor growth and/or induction. Thus inhibition of PRDX3 expression or activity is an appropriate means of inhibiting tumor growth. Moreover, anti-cancer agents can be screened and developed using the direct binding of cMyc to specific sequences of PRDX3.

Antisense constructs, antisense oligonucleotides, RNA interference constructs or siRNA duplex RNA molecules can be used to interfere with expression of PRDX3. Typically at least 15, 17, 19, or 21 nucleotides of the complement of PRDX3 mRNA sequence are sufficient for an antisense molecule. Typically at least 19, 21, 22, or 23 nucleotides of PRDX3 are sufficient for an RNA interference molecule. Preferably an RNA interference molecule will have a 2 nucleotide 3′ overhang. If the RNA interference molecule is expressed in a cell from a construct, for example from a hairpin molecule or from an inverted repeat of the desired PRDX3 sequence, then the endogenous cellular machinery will create the overhangs. siRNA molecules can be prepared by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

Antisense or RNA interference can be delivered in vitro to tumor cells or in vivo to tumors in a mammal. Typical delivery means known in the art can be used. For example, delivery to a tumor can be accomplished by intratumoral injections. Other modes of delivery can be used without limitation, including: intravenous, intramuscular, intraperitoneal, intraarterial, subcutaneous, and per os. Conversely in a mouse model, the antisense or RNA interference can be adminstered to a tumor cell in vitro, and the tumor cell can be subsequently administered to a mouse. Vectors can be selected for the desirable properties for any particular application. Vectors can be viral or plasmid. Non-viral carriers such as liposomes or nanospheres can also be used.

Drug discovery can be facilitated using the binding interaction of cMyc protein and PRDX3 DNA. Many different types of binding assays are known in the art; any of these can be used as is convenient and appropriate for the purpose. Briefly, these include reporter gene type assays, where a reporter gene (such as luciferase, chloramphenicol acetyl transferase, beta-galactosidease) is fused to the portion of PRDX3 which contains the binding sites. If a test substance is added and it reduces the expression of the reporter gene, then the test substance is identified as a potential anti-cancer drug because it appears to be interfering with the binding of cMyc to PRDX3 binding sites.

Double-stranded DNA fragments which comprise a cMyc-specific DNA binding site derived from PRDX3 genomic DNA can be attached to an insoluble polymeric support. The support may be agarose, cellulose, polycarbonate, polystyrene and the like. Such supported fragments may be used in screens to identify compounds which inhibit binding of cMyc to its specific DNA binding sites. Such inhibitors are potential chemotherapeutic agents.

Although any method can be employed which utilizes the cMyc-specific DNA binding sites of the present invention, one particular method is mentioned here. According to one method a test compound is incubated with a supported cMyc-binding DNA fragment and cMyc. The amount of cMyc which binds to the supported DNA fragment is determined. This determination can be performed according to any means which is convenient. For example, the amount of cMyc which can be removed after incubation with the supported fragment can be compared to the amount originally applied. Alternatively, the cMyc can be labeled and the amount which binds to the supported fragment can be assayed directly. If unsupported DNA fragments are used, then immunoprecipitation with anti-cMyc antibodies can be used to separate bound from unbound DNA fragments. In such a configuration the DNA can be labeled to facilitate quantitation of bound DNA.

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of PRDX3 gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of PRDX3 gene expression can be obtained by designing antisense oligonucleotides that will form duplexes to the control, 5′, or regulatory regions of the PRDX3 gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes. See WO 01/98340.

The invention provides assays for screening test compounds that bind to or modulate the activity of human PRDX3. A test compound preferably binds to a human PRDX3 polypeptide. More preferably, a test compound decreases or increases enzymatic activity by at least about 10, preferably about 50, more preferably about 75, 90, or 100% relative to the absence of the test compound.

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sc U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

Test compounds can be screened for the ability to inhibit PRDX3 activity using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Enzyme activity of PRDX3 can be determined according to any method known in the art. See for example Chae et al., Diabetes Res Clin Pract 1999 September; 45(2-3):101-12; Chae et al., Methods Enzymol 1999; 300:219-26.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLES

We sought to determine whether PRDX3 was a bona fide cMyc target gene by using Northern analysis of PRDX3 in several model systems. By using chromatin immunoprecipitation (ChIP), we also have examined the occupancy of Myc at multiple sites within the PRDX3 genomic sequence during serum stimulation of 2091 primary human fibroblasts. Then, we evaluated whether PRDX3 has a functional role in Myc-mediated cellular phenotypes. Deregulated c-myc expression induces cell-cycle progression (14), cellular proliferation, anchorage-independent growth (15), and apoptosis after withdrawal of serum (16) or glucose (17). In an effort to establish whether PRDX3 expression affects Myc-induced transformation, we generated stable Rat1a-myc fibroblast cell lines expressing murine PRDX3 in either the sense or antisense (AS) conformation. These cell lines then were evaluated in proliferation and apoptosis assays. To apply our findings to other cell systems, we chose the MCF7/ADR human breast cancer epithelial cell line (18) for further study of PRDX3. Our results demonstrate that c-Myc directly activates expression of a mitochondrial peroxiredoxin that is required for Myc-mediated transformation.

### Example 1

Northern Blotting. Northern blot analysis was performed as described (5). Blots were analyzed and quantitated on a PhosphorImager (Molecular Dynamics). The murine PRDX3 cDNA probe was obtained from IMAGE clone 577524. For R1a and R1a-myc cells, RNA was collected from logarithmically growing cells (adherent) or from cells grown in suspension for 48 h over a layer 0.7% agarose in DMEM (nonadherent). The blot was hybridized simultaneously with probes for PRDX3 and rpL32 (5). For in vivo analysis of PRDX3 expression, total RNA was isolated from mouse liver at 3, 4, and 5 days after adenoviral injection, as described (19). Twenty μg of RNA was loaded for each sample. Analysis of PRDX3 expression in 2091 primary human fibroblasts was performed by placing 50% confluent 2091 cells (American Type Culture Collection) in media containing 0.1% serum. After 48 h, confluent cells were stimulated with DMEM containing 10% (vol/vol) serum, and RNA was collected at the indicated time points. Northern blots containing 10 μg of RNA were probed with either human c-myc or PRDX3. The PRDX3 and c-myc signals were normalized to the ethidium bromide-stained gel of 18S RNA, which was quantitated with LABWORKS image analysis software (Ultraviolet Products).

### Example 2

Chromatin Immunoprecipitation. Quiescent human primary 2091 fibroblasts were serum stimulated for 0 or 2 h. ChIP was performed with a-Myc antibody (Santa Cruz Biotechnology, sc-764), as described (20). For PCR, 1/100th of the immunoprecipitate was used. PCR primers are given in Table 1, which is published as supporting information on the PNAS web site, www.pnas.org, and were designed by using the human PRDX3 genomic DNA sequence from the GenBank database (contig NT 008902). Real-time PCR was performed by using Sybr Green PCR core reagents (Applied Biosystems) according to the kit protocol (fragments D, F, G, I) or with 1×PCR buffer (Invitrogen), 2.5 mM MgCl2, 0.2 mM dNTPs, 1.25 units of Platinum Taq (Invitrogen), 0.5 μM primers, and 1×Sybr Green buffer (fragments A, B, C, E, H). Absolute quantitation of Myc-bound chromatin was performed by comparing the cycle threshold of each ChIP product to a standard curve generated with known amounts of total-input genomic DNA. Each reaction was analyzed within the linear range, and reactions were performed in triplicate. Plasmids. Murine PRDX3 cDNA was obtained from IMAGE consortium clone 577524. pSG5-PRDX3 and pSG5-PRDX3AS were created by cloning the Klenow-filled NotI-EcoRI fragment of 577524 into the Klenow-filled EcoRI site of pSG5 (Stratagene). Human PRDX3 cDNA was obtained from IMAGE consortium clone 50888. pSG5-PRDX3 and pSG5-PRDX3AS were created by NotI digestion of clone 50888 followed by partial digestion with HindIII. The 1.5-kb fragment corresponding to PRDX3 was filled with Klenow and cloned into the blunt Klenow-filled EcoRI site of pSG5. Constructs were screened for orientation and sequenced. Stable Transfectants. Stable pooled cell lines were generated by cotransfection of pSG5, pSG5-PRDX3, or pSG5-PRDX3AS with the puromycin resistance plasmid pBabe-puro (21) by using Lipofectamine (GIBCO) according to the manufacturer's instructions.

### Example 3

Immunoblotting. Immunoblotting was performed as described (5). Polyclonal rabbit antipeptide antibodies to murine PRDX3 were generated against amino acids 80-95 of murine PRDX3 (Research Genetics, Huntsville, Ala.). Polyclonal rabbit antipeptide antibodies to human PRDX3 were generated against amino acids 241-256 of human PRDX3 (Zymed). Monoclonal β-actin antibody was from Sigma (A-5441).

### Example 4

Growth and Transformation Assays. Growth curves were generated by plating triplicate samples for each cell line at an initial density of 5×103 cells per sample for R1a-myc cells or 1×104 cells per sample for MCF7/ADR cells. Live cells were counted by using a hemocytometer. The average cell number was plotted, curve fits were used to calculate doubling times, and R2 values were greater than 0.97 in each case. Methylcellulose assays consisted of four 35-mm dishes per cell line, at a density of 2×103 cells per dish, plated in 1 ml of 1.3% methylcellulose in DMEM. Photomicrographs were taken after 8 days (pSG5 and PRDX3) or 16 days (PRDX3AS). Colonies of all sizes from two experiments were counted after 7 days (pSG5 and PRDX3) or 14 days (PRDX3AS) to adjust for differences in doubling time.

### Example 5

Nude Mouse Assays. Cells (5×106) in 200 μl of sterile PBS were injected s.c. into the right flank of male homozygous nude mice at 6 weeks of age. Tumors were allowed to establish until the estimated tumor mass exceeded 1,500 mg. Experiments were approved by the Johns Hopkins School of Medicine Animal Care and Use Committee. Flow Cytometric Analyses. For apoptosis assays, cells were seeded at 5×105 per 10-cm2 plate and exposed to either media containing 0.1% serum or glucose-free media for 24 h. Cells were collected and stained with 5 μg/ml propidium iodide and FITC-conjugated annexin V (BioSource International, Camarillo, Calif.), followed by analysis using a Coulter EPICS 752 flow cytometer. All annexin V positive cells were included for statistical analysis. For fluorescence activated cell sorter (FACS) analysis of reactive oxygen species, mitochondrial membrane potential, and mitochondrial mass, cells were seeded at 5×105 per 10-cm2 plate and incubated at 37° C. in 5% CO2 for 30 min in the presence of 5 mg/ml 2′,7′-dichlorodihydrofluorescein diacetate (DCFH-DA), 20 nM DiOC6, or 100 nM NAO, respectively, all from Molecular Probes. Cells were washed with PBS, trypsinized, and resuspended. Cells incubated with DCFH-DA were resuspended in ice-cold media containing 5 mg/ml DCFH-DA and maintained on ice until analysis. Cells in DiOC6 were resuspended in 37° C. media and analyzed immediately. NAO-labeled cells were resuspended in 37° C. media containing 100 nM NAO and analyzed immediately. All analyses were performed with a Becton-Dickinson FACScan flow cytometer with a 488-nm argon laser. All analyses were performed at least three times, and a representative histogram is shown.

### Example 6

Electron Microscopy. Adherent cells were embedded by using the Pelco Eponate 12 kit (Ted Pella, Inc., Redding, Calif.). Then, cells were sectioned, followed by staining with uranyl acetate and lead citrate. Analysis was performed by using transmission electron microscopy.

### Example 7

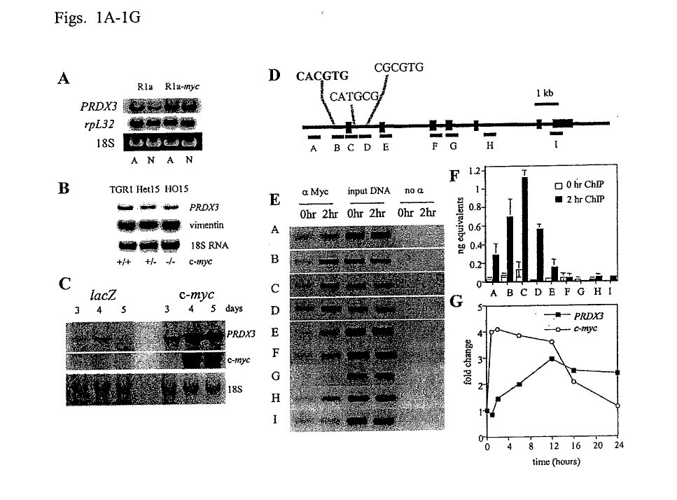

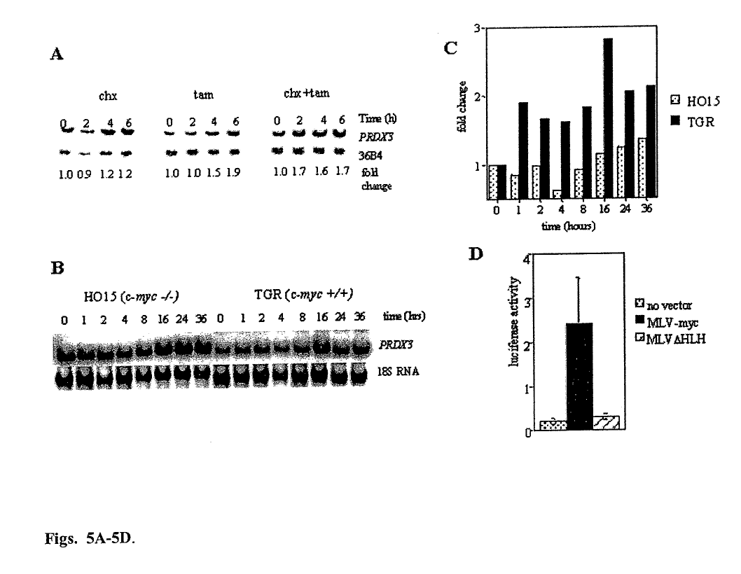

Analysis of PRDX3 Expression in Response to c-Myc. Northern analysis confirms the results of the original RDA screen, as shown in FIG. 1A. PRDX3 is two-fold more highly expressed in adherent R1a-myc cells relative to R1a cells, with the difference in expression becoming six-fold when the cells are nonadherent. Because R1a cells growth-arrest when they are not attached to a substrate while R1a-myc cells continue to proliferate (5), the original RDA screen cannot distinguish between direct c-Myc target genes and genes that are growth-related, non-Myc targets. Therefore, we used the Rat1MycER system (14, 22) to determine whether Myc directly activates PRDX3. This system utilizes a fusion of c-myc to the hormone-binding domain of the estrogen receptor. The fusion protein is retained in the cytosol until the addition of tamoxifen, an estrogen analog, whereupon the protein translocates to the nucleus and activates its biological targets. Cycloheximide is used to inhibit protein synthesis, thereby allowing identification of genes that are directly activated by Myc. FIG. 5A, which is published as supporting information on the PNAS web site, shows that PRDX3 expression increases in the presence of both cycloheximide and tamoxifen, suggesting that c-Myc directly activates transcription of PRDX3. Examination of logarithmically growing c-myc-null fibroblasts (23) indicates that PRDX3 expression is decreased by 50% in the absence of myc (FIG. 1B). PRDX3 expression is also induced during serum stimulation of quiescent c-myc+/+ cells (FIGS. 5 B and C). PRDX3 expression increases after 1 h of serum stimulation and reaches a maximum of 2.8-fold after 16 h. However, only a 1.3-fold increase is seen after serum stimulation of c-myc−/− cells. These results indicate that PRDX3 is a c-Myc responsive gene and that PRDX3 expression is minimally induced by serum in the absence of Myc.

A recently described in vivo model of transient c-Myc overexpression (19) also indicates that c-Myc regulates PRDX3. Mice injected with adenoviral c-myc show a dramatic increase in hepatic PRDX3 expression, whereas mice injected with control LacZ adenovirus show a minimal increase in PRDX3 (FIG. 1C). The increase in PRDX3 expression parallels that of c-myc. To determine whether Myc binds directly to PRDX3 in vivo, we performed chromatin immunoprecipitation during serum stimulation of primary human fibroblasts. Scanning analysis of the 11-kb genomic PRDX3 sequence (FIG. 1D) indicates that Myc binds to a region containing the sole canonical E box 179 bp upstream from the translational start site, as well as two noncanonical E boxes within the first intron of PRDX3 (FIG. 1E). Quantitative real-time PCR analysis of PRDX3 when Myc levels are low, at 0 h, indicates that most fragments exhibit a similar level of binding (FIG. 1F, white bars). At 2 h, Myc binds fragments B, C, and D preferentially, with fragment C showing a 22-fold increase in binding relative to negative distal sites F, H, and I (FIG. 1F, black bars). Despite the presence of multiple noncanonical E boxes located throughout the genomic PRDX3 sequence, Myc binds specifically within a 930-bp region, spanning fragments B, C, and D at the 5′ end of PRDX3. Northern blot analysis during serum stimulation of 2091 fibroblasts indicates that myc expression is maximal between 1-2 h (FIG. 1G). Expression of PRDX3 is induced after 2 h and reaches a maximum at 12 h. Taken together, our results establish that Myc binds directly to PRDX3 in vivo and activates transcription.

### Example 8

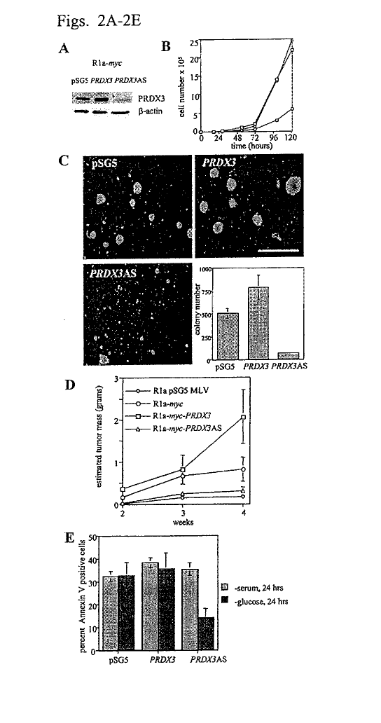

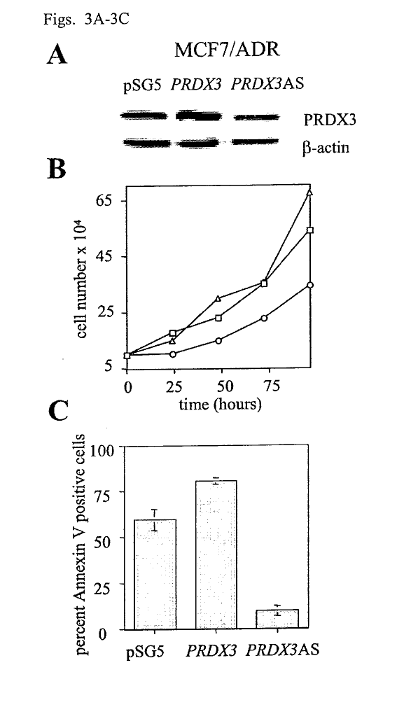

Effect of PRDX3 on Proliferation and Apoptosis in Rat1a-myc Cells. To determine the role of PRDX3 in Myc-mediated transformation, we generated pooled R1a-myc fibroblast cell lines stably expressing murine PRDX3 in either the sense or AS conformation (FIG. 2A). Characterization of the growth rate of these cells shows a decrease in the growth rate of R1a-myc-PRDX3AS cells, whereas control and R1a-myc-PRDX3 cells display similar doubling times (FIG. 2B). This decrease in growth rate is not caused by increased apoptosis, as staining with annexin V is nearly identical among the three cell lines (data not shown). Because PRDX3 was originally identified in a screen under conditions of anchorage-independent growth, we hypothesized that PRDX3 would affect colony formation in semisolid media. FIG. 2C demonstrates that R1a-myc-PRDX3 cells form colonies at a higher frequency than pSG5 control cells, whereas cells with PRDX3AS form very few colonies. To determine whether our observations applied in vivo, we injected these same cells into nude mice (FIG. 2D). R1a-myc cells expressing AS PRDX3 did not readily form tumors and were only slightly more tumorigenic than R1a cells expressing control vectors alone (R1a pSG5 MLV). In contrast, R1a-myc cells overexpressing PRDX3 formed larger tumors than R1a-myc cells, suggesting that elevated PRDX3 expression confers a growth advantage in vivo. These results indicate that PRDX3 affects both growth rate and transformation in R1a-myc cells. We also used these cell lines to examine Myc-induced apoptosis after serum or glucose deprivation. We found that PRDX3 expression does not affect apoptosis after serum deprivation (FIG. 2E, light bars). However, PRDX3 does affect apoptosis after glucose withdrawal (FIG. 2E, dark bars). Cells expressing AS PRDX3 are resistant to apoptosis after removal of glucose, whereas cells with increased PRDX3 remain sensitive to glucose deprivation-induced apoptosis. Effect of PRDX3 on Proliferation and Apoptosis in MCF7/ADR Cells. To demonstrate that our findings were not specific to R1a-myc cells, we chose the MCF7/ADR human breast cancer epithelial cell line (18). This cell line undergoes extensive apoptosis after glucose withdrawal, and apoptosis can be inhibited by reduction of c-Myc expression with AS oligonucleotides (24). Apoptosis depends on the formation of oxygen radicals, as inhibition of oxygen radical formation using the free radical scavenger sodium pyruvate is sufficient to inhibit apoptosis (25). By using full-length human PRDX3 cDNA, we generated stable pooled cell lines that either overexpress or show decreased levels of human PRDX3 protein (FIG. 3A). Analysis of the growth rate of these cells shows that PRDX3AS cells show a decreased growth rate relative to control cells, although the result is less dramatic than that for R1a-myc cells (FIG. 3B). We also assayed apoptosis after glucose deprivation for 24 h. Cells that overexpress PRDX3 show a reproducible increase in apoptosis, whereas cells with diminished PRDX3 are resistant to apoptosis (FIG. 3C). These results confirm that PRDX3 is required for proliferation in transformed cells, and that AS PRDX3 inhibits apoptosis after glucose deprivation.

### Example 9

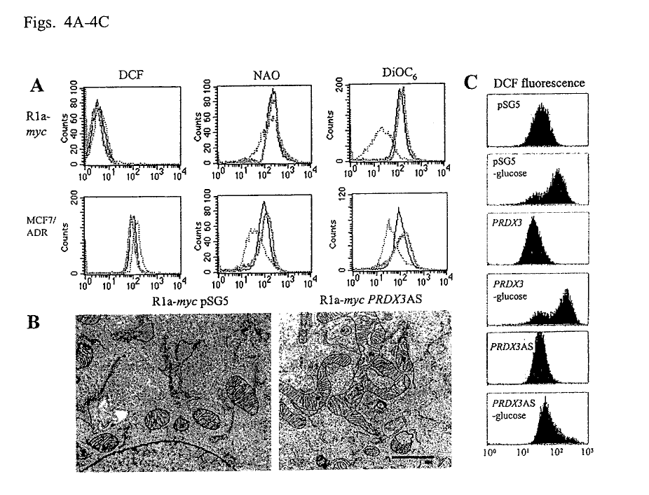

Effect of PRDX3 on Mitochondrial Function and Structure. Because PRDX3 localizes to mitochondria, we examined several parameters that reflect mitochondrial integrity and function. FIG. 4A demonstrates that MCF7/ADR cells expressing PRDX3AS show increased levels of reactive oxygen species as measured by the redox-sensitive dye DCFH-DA (26), which is oxidized to fluorescent DCF. However, R1a-myc-PRDX3AS cells show a minimal increase in reactive oxygen species. Analysis of mitochondrial mass with 10-N-nonyl-acridine orange (NAO) (27) reveals that MCF7/ADR-PRDX3AS cells show decreased mitochondrial mass, whereas R1a-myc-PRDX3AS cells also show a small percentage of cells with reduced mitochondrial mass. Reduction of PRDX3 in both cell lines results in a decrease in mitochondrial membrane potential, indicated by the reduced uptake of 3,3′-dihexyloxacarbocyanine iodide (DiOC6), as shown in FIG. 4A. Although PRDX3AS cells have a lower mitochondrial mass and would, therefore, be expected to show reduced uptake of DiOC6, the membrane potential is diminished even after the mitochondrial mass is taken into account (data not shown). In addition to functional defects, we also observed severe morphological defects when using electron microscopy. R1a-myc-PRDX3AS cells show distorted mitochondrial architecture, with elongated mitochondria displaying branched or circular lobes (FIG. 4B). Analysis of 10 individual R1a-myc-pSG5 cells indicates that although some cells also had longer mitochondria, none of the cells showed branched or looped mitochondria. In contrast, 9 of 10 R1a-myc-PRDX3AS cells showed branched mitochondria, and 3 of 9 also showed looped mitochondria. We hypothesized that the reduced mitochondrial membrane potential could prevent the generation of reactive oxygen species (ROS) during glucose deprivation-induced apoptosis. Previously, it had been shown that the mitochondrial membrane potential is required for generating ROS in bovine aortic endothelial cells after exposure to hyperglycemia (28). Analysis of MCF7/ADR cells after glucose withdrawal demonstrates that PRDX3AS cells show a minimal increase in ROS, whereas control cells show a dramatic increase in ROS (FIG. 4C).

### Example 10

Our data suggest that one of the primary defects in PRDX3AS cells is a reduced mitochondrial membrane potential. The reduction in membrane potential may be a result of oxidative damage to components of the respiratory chain complexes (29), which, in turn, would disrupt the proton gradient across the inner mitochondrial membrane. A recent report attributing peroxynitrite reductase activity to bacterial peroxiredoxins (30) suggests peroxynitrite as a possible mediator of inhibition of respiratory chain activity and reduction of mitochondrial membrane potential (31). Our results are consistent with the observation that reduced levels of another mitochondrial antioxidant, MnSOD, also result in mitochondrial dysfunction and reduced mitochondrial membrane potential (32). Several reports underscore the potential significance of nuclear c-Myc target genes whose protein products localize to mitochondria. In one case, the mycER system was used to analyze thousands of genes on microarrays (4). Three genes with protein products that localize to mitochondria, peptidyl-prolyl cis-trans isomerase F, heat shock protein 60, and the chaperone grpE were identified by using this technique. Mother study focused on genes regulated by myc-induced lymphomagenesis in the bursa of Fabricius (33). Several mitochondrial genes, including matrix nucleoside diphosphate kinase and matrix protein P1, were identified. Additionally, recent evidence suggests that the response of Myc to diverse apoptotic stimuli converges at a common mitochondrial signaling element (34). Microarray analysis comparing c-myc+/+ and c-myc−/− cells supports our conclusion that PRDX3 is a c-Myc target gene (3). Rat PRDX3, termed thioredoxin peroxidase, was identified as a gene that was more highly expressed in both wild-type fibroblasts and myc−/− fibroblasts with reconstituted c-myc as compared with c-myc−/− cells. This same study also found that PRDX3 expression was increased 2.4-fold upon expression of ectopic myc in normal c-myc+/+fibroblasts. This finding indicates that deregulated overexpression of myc, which mimics conditions found in cancer cells, induces PRDX3, and it suggests that at least some of the target genes that are regulated by myc under physiological conditions are also activated when myc is overexpressed. We hypothesize that Myc is not the sole regulator of PRDX3 expression, as PRDX3 is still expressed in myc−/− fibroblasts. Rather, PRDX3 likely belongs to a class of genes that facilitates accelerated cellular growth and metabolism induced by c-Myc. The mechanism by which c-Myc regulates both proliferation and apoptosis remains unclear. The c-Myc target gene ODC has been found to affect both processes, in that overexpression stimulates apoptosis, whereas inhibiting ODC activity blocks cell-cycle progression (35). This observation led to the multiple-effector model, whereby c-Myc regulates targets that overlap in function. In support of this model, our data indicate that Myc regulates a mitochondrial peroxiredoxin that is required for proliferation as well as apoptosis in transformed cells. Additionally, our results suggest that reduced mitochondrial function affects Myc-mediated transformation. Although is has been reported that the loss of mitochondrial membrane potential is a downstream event in Myc-mediated apoptosis (36), it is not known how the mitochondrial membrane potential affects proliferation and the apoptotic signaling cascade. Recently, it has been suggested that the mitochondrial membrane potential could be an integrator of growth, maturation, and apoptotic pathways (37). The observation that both transformation and apoptosis are affected in PRDX3AS cells supports this hypothesis.

### Example 11

Primer sequences used for ChIP analysis

| | | SEQ ID | | SEQ ID |
|---|---|---|---|---|
| Fragment | 5′ primer | NO: | 3′ primer | NO: |
| A | 5′-tactcatgaagctcaggcag- | 3 | 5′-tgacaaattgcagtcttgga- | 12 |
| | 3′ | | 3′ | |
| B | 5′- | 4 | 5′-ccctttaaggctgaatgctt- | 13 |
| | cctggattcgttcttttaaggttgg- | | 3′ | |
| | 3′ | | | |
| C | 5′-tggagacactggtggctccg- | 5 | 5′- | 14 |
| | 3′ | | agtctgagaaaggcgaaggc- | |
| | | | 3′ | |
| D | 5′-gccttcgcctttctcagact-3′ | 6 | 5′-gccaccgcactctgccggtt- | 15 |
| | | | 3′ | |
| E | 5′-cagggacagctgaaaccacc- | 7 | 5′- | 16 |
| | 3′ | | cagagcccctgtccagagac-3′ | |
| F | 5′-catgccatgcacctgctgtc-3′ | 8 | 5′-acaagctacagatcccagct- | 17 |
| | | | 3′ | |
| G | 5′-ctgtgaagttgtcgcagtct-3′ | 9 | 5′-gtttacctgtaaccccagct- | 18 |
| | | | 3′ | |
| H | 5′-ggccacactgctccatactc-3′ | 10 | 5′-atcctaacaactgctgccag- | 19 |
| | | | 3′ | |
| I | 5′-tcagatcaagccaagtccag- | 11 | 5′-ctgtagaaactagctagcca- | 20 |
| | 3′ | | 3′ | |

### REFERENCES

Henriksson, M. & Luscher, B. (1996). Adv. Cancer Res. 68, 109182.
Schuhmacher, M., Kohlhuber, F., Holzel, M., Kaiser, C., Burtscher, H., Jarsch, M., Bornkamm, G. W., Laux, G., Polack, A., Weidle, U. H., & Eick, D. (2001). Nucleic Acids Res. 29, 397-406.
Guo, Q. M., Malek, R. L., Kim, S., Chiao, C., He, M., Ruffy, M., Sanka, K., Lee, N. H., Dang, C. V., & Liu, E. T. (2000). Cancer Res. 60, 5922-5928.
Coller, H. A., Grandori, C., Tamayo, P., Colbert, T., Lander, E. S., Eisenman, R. N., & Golub, T. R. (2000). Proc. Natl. Acad. Sci. USA 97, 3260-3265.
Lewis, B. C., Shim, H., Li, Q., Wu, C. S., Lee, L. A., Maity, A., & Dang, C. V. (1997). Mol. Cell. Biol. 17, 4967-4978.
Yamamoto, T., Matsui, Y., Natori, S., & Obinata, M. (1989). Gene 80, 337-343.
Tsuji, K., Copeland, N. G., Jenkins, N. A., & Obinata, M. (1995). Biochem. J. 307, 377-381.
Chae, H. Z., Chung, S. J., & Rhee, S. G. (1994). J. Biol. Chem. 269, 27670-27678.
Kang, S. W., Chae, H. Z., Seo, M. S., Kim, K., Baines, I. C., & Rhee, S. G. (1998). J. Biol. Chem. 273, 6297-6302.
Prosperi, M. T., Ferbus, D., Karczinski, I., & Goubin, G. (1993). J. Biol. Chem. 268, 11050-11056.
Zhang, P., Liu, B., Kang, S. W., Seo, M. S., Rhee, S. G., & Obeid, L. M. (1997). J. Biol. Chem. 272, 30615-30618.
Araki, M., Nanri, H., Ejima, K., Murasato, Y., Fujiwara, T., Nakashima, Y., & Ikeda, M. (1999). J. Biol. Chem. 274, 2271-2278.
Noh, D. Y., Alin, S. J., Lee, R. A., Kim, S. W., Park, I. A., & Chae, H. Z. (2001). Anticancer Res. 21, 2085-2090.
Eilers, M., Picard, D., Yamamoto, K. R., & Bishop, J. M. (1989). Nature (London) 340, 66-68.
Stone, J., de Lange, T., Ramsay, G., Jakobovits, E., Bishop, J. M., Varmus, H., & Lee, W. (1987). Mol. Cell. Biol. 7, 1697-1709.
Evan, G. I., Wyllie, A. H., Gilbert, C. S., Littlewood, T. D., Land, H., Brooks, M., Waters, C. M., Penn, L. Z., & Hancock, D. C. (1992). Cell 69, 119-128.
Shim, H., Chun, Y. S., Lewis, B. C., & Dang, C. V. (1998). Proc. Natl. Acad. Sci. USA 95, 1511-1516.
Fairchild, C. R., Ivy, S. P., Kao-Shan, C. S., Whang-Peng, J., Rosen, N., Israel, M. A., Melera, P. W., Cowan, K. H., & Goldsmith, M. E. (1987). Cancer Res. 47, 5141-5148.
Kim, S., Li, Q., Dang, C. V., & Lee, L. A. (2000). Proc. Natl. Acad. Sci. USA 97, 11198-11202.
Boyd, K. E., Wells, J., Gutman, J., Bartley, S. M., & Farnham, P. J. (1998). Proc. Natl. Acad. Sci. USA 95, 13887-13892
Egan, S. E., Giddings, B. W., Brooks, M. W., Buday, L., Sizeland, A. M., & Weinberg, R. A. (1993). Nature (London) 363, 45-51.
Littlewood, T. D., Hancock, D. C., Danielian, P. S., Parker, M. G., & Evan, G. I. (1995). Nucleic Acids Res. 23, 1686-1690.
Mateyak, M. K., Obaya, A. J., Adachi, S., & Sedivy, J. M. (1997). Cell. Growth Differ. 8, 1039-1048.
Lee, Y. J., Galoforo, S. S., Berns, C. M., Tong, W. P., Kim, H. R., & Corry, P. M. (1997). J. Cell Sci. 110, 681-686.
Lee, Y. J., Galoforo, S. S., Berns, C. M., Chen, J. C., Davis, B. H., Sim, J. E., Corry, P. M., & Spitz, D. R. (1998). J. Biol. Chem. 273, 5294-5299.
Hockenbery, D. M., Oltvai, Z. N., Yin, X. M., Milliman, C. L., & Korsmeyer, S. J. (1993). Cell 75, 241-251.
Petit, J. M., Maftah, A., Ratinaud, M. H., & Julien, R. (1992). Eur. J. Biochem. 209, 267-273.
Nishikawa, T., Edelstein, D., Du, X. L., Yamagishi, S., Matsumura, T., Kaneda, Y., Yorek, M. A., Beebe, D., Oates, P. J., Hammes, H. P., et al. (2000). Nature (London) 404, 787-790
Zhang, Y., Marcillat, O., Giulivi, C., Ernster, L., & Davies, K. J. (1990). J. Biol. Chem. 265, 16330-16336.
Bryk, R., Griffin, P., & Nathan, C. (2000). Nature (London) 407, 211-215.
Brown, G. C. (1999). Biochim. Biophys. Acta 1411, 351-369.
Kokoszka, J. E., Coskun, P., Esposito, L. A., & Wallace, D. C. (2001). Proc. Natl. Acad. Sci. USA 98, 2278-2283.
Neiman, P. E., Ruddell, A., Jasoni, C., Loring, G., Thomas, S. J., Brandvold, K. A., Lee, R., Burnside, J., & Delrow, J. (2001). Proc. Natl. Acad. Sci. USA 98, 6378-6383.
Soucie, E. L., Annis, M. G., Sedivy, J., Filmus, J., Leber, B., Andrews, D. W., & Penn, L. Z. (2001). Mol. Cell. Biol. 21, 4725-4736.
Packham, G., Porter, C. W., & Cleveland, J. L. (1996). Oncogene 13, 461-469.
Hotti, A., Jarvinen, K., Siivola, P., & Holtta, E. (2000). Oncogene 19, 2354-2362.
Augenlicht, L. H. & Heerdt, B. G. (2001). Nat. Genet. 28, 104-105.
Grandori, C., Mac, J., Siebelt, F., Ayer, D. E., & Eisenman, R. N. (1996). EMBO J. 15, 4344-4357.
Blackwell, T. K., Huang, J., Ma, A., Kretzner, L., Alt, F. W., Eisenman, R. N., & Weintraub, H. (1993). Mol. Cell. Biol. 13, 5216-5224.

1. A method comprising:
delivering to a tumor cell an antisense construct comprising at least 15 nucleotides of a murine or human PRDX3 cDNA, whereby the tumor cell expresses an mRNA molecule which is complementary to native PRDX3 mRNA.

2. The method of claim 1 wherein the cDNA is human.

3. The method of claim 1 wherein the cDNA is murine.

4. The method of claim 1 wherein the tumor cell is in a mammal.

5. The method of claim 4 wherein the antisense construct is delivered by intratumoral injection.

6. The method of claim 4 wherein the antisense construct is delivered to the tumor cell in vitro, and the tumor cell is thereafter injected into a nude mouse.

7. A method comprising:
delivering to a tumor cell an RNA interference construct comprising at least 19 nucleotides of a murine or human PRDX3 cDNA, whereby the tumor cell expresses a double stranded RNA molecule one of whose strands is complementary to native PRDX3 mRNA.

8. The method of claim 7 wherein the cDNA is human.

9. The method of claim 7 wherein the cDNA is murine.

10. The method of claim 7 wherein the tumor cell is in a mammal.

11. The method of claim 10 wherein the RNA interference construct is delivered by intratumoral injection.

12. The method of claim 10 wherein the RNA interference construct is delivered to the tumor cell in vitro, and the tumor cell is thereafter injected into a nude mouse.

13-14. (canceled)

15. The method of claim 7 wherein the construct contains an inverted repeat of the PRDX3 cDNA.

16. A method comprising:
delivering to a tumor cell siRNA comprising 19 to 21 bp duplexes of a murine or human PRDX3 mRNA with 2 nt 3′ overhangs, whereby PRDX3 mRNA produced by the tumor cell is cleaved.

17. The method of claim 16 wherein the mRNA is human.

18. The method of claim 16 wherein the mRNA is murine.

19. The method of claim 16 wherein the tumor cell is in a mammal.

20. The method of claim 19 wherein the siRNA is delivered by intratumoral injection.

21. The method of claim 19 wherein the siRNA is delivered to the tumor cell in vitro, and the tumor cell is thereafter injected into a nude mouse.

22.-33. (canceled)

34. A method comprising:
delivering to a tumor cell an inhibitor of peroxiredoxin 3 activity.


SEQUENCE LISTING (24 sequences)

SEQ ID NO: 1 - DNA, 29 bp, Homo sapiens
tgcccggggacacagtaatccacacaagg

SEQ ID NO: 2 - DNA, 28 bp, Homo sapiens
tgctcgaggccaccgcactctgccggtt

SEQ ID NO: 3 - DNA, 20 bp, Homo sapiens
tactcatgaagctcaggcag

SEQ ID NO: 4 - DNA, 25 bp, Homo sapiens
cctggattcgttcttttaaggttgg

SEQ ID NO: 5 - DNA, 20 bp, Homo sapiens
tggagacactggtggctccg

SEQ ID NO: 6 - DNA, 20 bp, Homo sapiens
gccttcgcctttctcagact

SEQ ID NO: 7 - DNA, 20 bp, Homo sapiens
cagggacagctgaaaccacc

SEQ ID NO: 8 - DNA, 20 bp, Homo sapiens
catgccatgcacctgctgtc

SEQ ID NO: 9 - DNA, 20 bp, Homo sapiens
ctgtgaagttgtcgcagtct

SEQ ID NO: 10 - DNA, 20 bp, Homo sapiens
ggccacactgctccatactc

SEQ ID NO: 11 - DNA, 20 bp, Homo sapiens
tcagatcaagccaagtccag

SEQ ID NO: 12 - DNA, 20 bp, Homo sapiens
tgacaaattgcagtcttgga

SEQ ID NO: 13 - DNA, 20 bp, Homo sapiens
ccctttaaggctgaatgctt

SEQ ID NO: 14 - DNA, 20 bp, Homo sapiens
agtctgagaaaggcgaaggc

SEQ ID NO: 15 - DNA, 20 bp, Homo sapiens
gccaccgcactctgccggtt

SEQ ID NO: 16 - DNA, 20 bp, Homo sapiens
cagagcccctgtccagagac

SEQ ID NO: 17 - DNA, 20 bp, Homo sapiens
acaagctacagatcccagct

SEQ ID NO: 18 - DNA, 20 bp, Homo sapiens
gtttacctgtaaccccagct

SEQ ID NO: 19 - DNA, 20 bp, Homo sapiens
atcctaacaactgctgccag

SEQ ID NO: 20 - DNA, 20 bp, Homo sapiens
ctgtagaaactagctagcca

SEQ ID NO: 21 - protein, 257 aa, Mus musculus
MAAAAGRLLWSSVARHASAISRSISASTVLRPVASRRTCLTDILWSASAQGKSAFSTSSSFHTPAVTQHAPYFKGTAVVNGEFKELSLDDFKGKYLVLFFYPLDFTFVCPTEIVAFSDKANEFHDVNCEVVAVSVDSHFSHLAWINTPRKNGGLGHMNITLLSDITKQISRDYGVLLESAGIALRGLFIIDPNGVVKHLSVNDLPVGRSVEETLRLVKAFQFVETHGEVCPANWTPESPTIKPSPTASKEYFEKVHQ

SEQ ID NO: 22 - DNA, 1382 bp, Mus musculus
ctactcctcggtatctccgcctatcgtgcctcttgcgtgctctgaagatggcggcagctgcgggaaggttgctctggtcctcggttgctcgtcatgcaagtgctatttcccggagtatttctgcctcaacagttcttaggcctgttgcttctagaagaacctgtttgacagacatactgtggtctgcctctgcccaaggaaagtcagcctttagcaccagttcctctttccacacccctgctgtcacccagcacgcgccctattttaaaggtactgctgttgtcaatggagagttcaaagagctgagtctcgacgactttaagggaaaatacttggtgcttttcttctaccctttggatttcacatttgtgtgtcctacagaaattgttgctttcagtgacaaagccaatgaatttcatgatgtaaactgtgaagtagttgcagtttcagtggattcccacttcagtcatcttgcctggatcaacacaccaagaaagaatggtggtttgggccacatgaacatcacactgttgtcggatataactaagcagatatcccgagactacggagtgctgttggaaagtgctggcattgcactcagaggtctcttcattattgaccctaatggtgtcgtcaagcacctgagtgtcaacgaccttccggtgggccgcagtgtggaagaaacactccgtttggtaaaggcgttccagtttgtagagacccatggagaagtctgcccagccaactggacaccagagtcccctacgatcaagccaagtccaacagcttccaaagagtactttgagaaggtccatcagtaggccatcctatgtctgcaattacctgaagcttttcaggccaaaaaagagccccagctggaatccttccaatgccttgaagattatttatagaatggcaaaacctcattatgtttgtgtttataagtactgctccacaggctttgtaattctaagacaggttcaggctctctaaaggtggctagctgcttccatagctgcccttactagggacttcttggtggctaaccaattctccccgagtgctttgcccccatttcttggatcatgtccttagagggtaagcattctttcccttagcctgccctgaaccttggtctacagtgaagtagcacatagtgccagtacttggtgaaatgaagtagcacatagcaccagcacttaatggaagcttctgatcaaggtcctaaaatttcctcttgaatttttgtgaattatgctgaatttccctttttttttttttaaacagtgtccttgtgtgttctgaggtattgaagaggtataatcatgaaggactatgtctaatccataagtcattttcttcaagagctggatatatagaat

SEQ ID NO: 23 - protein, 256 aa, Homo sapiens
MAAAVGRLLRASVARHVSAIPWGISATAALRPAACGRTSLTNLLCSGSSQAKLFSTSSSCHAPAVTQHAPYFKGTAVVNGEFKDLSLDDFKGKYLVLFFYPLDFTFVCPTEIVAFSDKANEFHDVNCEVVAVSVDSHFSHLAWINTPRKNGGLGHMNIALLSDLTKQISRDYGVLLEGSGLALRGLFIIDPNGVIKHLSVNDLPVGRSVEETLRLVKAFQYVETHGEVCPANWTPDSPTIKPSPAASKEYFQKVNQ

SEQ ID NO: 24 - DNA, 1542 bp, Homo sapiens
ctgaagatggcggctgctgtaggacggttgctccgagcgtcggttgcccgacatgtgagtgccattccttggggcatttctgccactgcagccctcaggcctgctgcatgtggaagaacgagcttgacaaatttattgtgttctggttccagtcaagcaaaattattcagcaccagttcctcatgccatgcacctgctgtcacccagcatgcaccctattttaagggtacagccgttgtcaatggagagttcaaagacctaagccttgatgactttaaggggaaatatttggtgcttttcttctatcctttggatttcacctttgtgtgtcctacagaaattgttgcttttagtgacaaagctaacgaatttcacgatgtgaactgtgaagttgtcgcagtctcagtggattcccactttagccatcttgcctggataaatacaccaagaaagaatggtggtttgggccacatgaacatcgcactcttgtcagacttaactaagcagatttcccgagactacggtgtgctgttagaaggttctggtcttgcactaagaggtctcttcataattgaccccaatggagtcatcaagcatttgagcgtcaacgatctcccagtgggccgaagcgtggaagaaaccctccgcttggtgaaggcgttccagtatgtagaaacacatggagaagtctgcccagcgaactggacaccggattctcctacgatcaagccaagtccagctgcttccaaagagtactttcagaaggtaaatcagtagatcacccatgtgtatctgcaccttctcaactgagagaagaaccacagttgaaacctgcttttatcattttcaagatggttatttgtagaaggcaaggaaccaattatgcttgtattcataagtattactctaaatgttttgtttttgtaattctggctaggaccttttaaacatggttagttgctagtacaggaatcgtttattggtaacatcttggtggctggctagctagtttctacagaacataatttgcctctatagaaggctattcttagatcatgtctcaatggaaacactcttctttcttagccttacttgaatcttgcctataataaagtagagcaacacacattgaaagcttctgatcaacggtcctgaaattttcatcttgaatgtctttgtattaaactgaattttcttttaagctaacaaagatcataattttcaatgattagccgtgtaactcctgcaatgaatgtttatgtgattgaagcaaatgtgaatcgtattattttaaaaagtggcagagtgacttaactgatcatgcatgatccctcatccctgaaattgagtttatgtagtcattttacttattttattcattagctaactttgtctatgtatatttctagatattgattagtgtaatcgattataaaggatatttatcaaatccagggattgcattttgaaattataattattttctttgctgaagtattcattgtaaaacatacaaataacatatttaaacaaaaaaaaaaa